(19) United States
(12) Patent Application Publication
(10) Pub. No.: US 2004/0082623 A1
(43) Pub. Date: Apr. 29, 2004

(54) Fmoc-l-leucine and derivatives thereof as ppar-gamma agonists

(76) Inventors: Stephane Rochhi, Villefrance-sur-Mer (FR); Johan Auwerx, Hindisheim (FR); Joseph Vamecq, Saint-Symphorien (BE)

Correspondence Address: FOLEY AND LARDNER, SUITE 500, 3000 K STREET NW, WASHINGTON, DC, 20007, US

(21) Appl. No.: 10/312,778

(22) PCT Filed: Jun. 28, 2001
(86) PCT No.: PCT/IB01/01581

(51) Int. Cl. A61K 31/44; A61K 31/198

(52) U.S. Cl. 514/357; 514/567; 514/561

(57) ABSTRACT

The present invention relates to a method for treating or preventing a PPAR-γ mediated disease or condition comprising administration of a therapeutically effective amount of FMOC-L-Leucine or derivatives thereof, of the formula I. Said method is particularly useful for treating or preventing anorexia, hyperlypidemia, insulin resistance, inflammatory diseases, cancer and sking disorders.

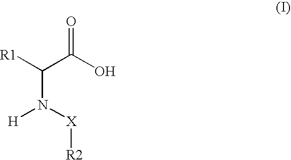

The present invention relates to a method for treating or preventing a PPAR-γ mediated disease or condition comprising administration of a therapeutically effective amount of FMOC-L-Leucine (N-(9-fluroroenylmethyloxycarbonyl)-L-Leucine) or derivatives thereof.

The peroxisome proliferator-activated receptors (PPARs) are nuclear hormone receptors which bind DNA as heterodimers with the retinoid X receptor (RXR) and activate a number of target genes, mainly involved in the control of lipid metabolism. PPARs have pleiotropic biological activities and wide-ranging medical applications, ranging from uses in metabolic disorders to eventual applications in inflammation, and cancer (Desvergne and Wahli, 1999; Schoonjans et al., 1997; Spiegelman and Flier, 1996). Especially, PPARγ has received a lot of attention because PPARγ-activating drugs represent a novel opportunity to treat type 2 diabetes. PPARγ can be activated by naturally occurring ligands, such as the long-chain fatty acid-derivatives, 15-deoxy-Δ12,14-prostaglandin J2, Δ12-prostaglandin J2 (PG J2), and 9- and 13-cis-hydroxyoctadecadienoic acid (HODE) (Forman et al., 1995; Kliewer et al., 1995; Nagy et al., 1998). Most interesting is, however, the observation that the anti-diabetic activity of a group of the glitazones, which all possess a thiazolidinedione ring (FIG. 1, Panel A), results from. their PPARγ activating properties (Berger et al., 1996; Willson et al., 1996). The therapeutic efficacy of the current thiazolidinediones (TZDs) in type 2 diabetes is, however, far from optimal and several undesirable side-effects have been reported for this drug class (Schoonjans and Auwerx, 2000). As a result, there is a need for non-TZD-based alternative ligands of PPARγ.

Recently, a series of L-tyrosine based PPARγ ligands were designed by replacing the thiazolidinedione ring with a carboxylic acid and by introducing an amine function or the adjacent carbon while keeping the parahydroxybenzyl sequence (FIG. 1, Design 1 route). An optimal PPARγ activity was obtained when the amine on the alpha carbon of the L-tyrosine ligands was substituted with a benzoylphenyl function, leading to the development of N-(2-benzoylphenyl)-L-tyrosine derivatives (FIG. 1, Panel B, left part) (Cobb et al., 1998; Collins et al., 1998). Rigidifying the benzoyl and phenyl moieties of this alpha-amino substituent through an additional phenyl-phenyl bond (FIG. 1, Panel B, right part) leads to compounds with good potencies (Cobb et al., 1998; Collins et al., 1998).

In connection with the present invention, it was unexpectedly found that FMOC-L-tyrosine derivatives were devoid of PPARγ activity, whereas FMOC-L-leucine (hereafter also designated as F-L-Leu), whose structure is lacking the parahydroybenzyl sequence present in both the TZDs and previously developed L-tyrosine-based PPARγ ligands (FIG. 1), is a new potent insulin-sensitizing compound with unique PPARγ-activating and -binding properties.

F-L-Leu, referred to as NPC 15199, has been described as a drug active in various inflammatory models through an unknown anti-inflammatory mechanism (Miller et al., 1993) (Burch et al., 1991). But, the present invention provides new applications of this compound and derivatives thereof as a PPARγ agonist.

### DESCRIPTION

The present invention relates to a method for treating or preventing a PPAR-γ mediated disease or condition comprising administration of a therapeutically effective amount of a compound having the formula I:

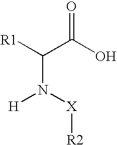

wherein R1 is selected from a linear or branched alkyl, alkenyl and alkynyl group comprising from 1 to 6 carbon atoms,

X is a chain comprising from 1 to 6 carbon atoms which may comprise one to four heteroatoms,

R2 is a condensed polycyclic group comprising at least two cycles.

In a first embodiment, the R2 group comprises at least two cycles selected from carbocycles and heterocycles.

The R2 group can be advantageously selected from

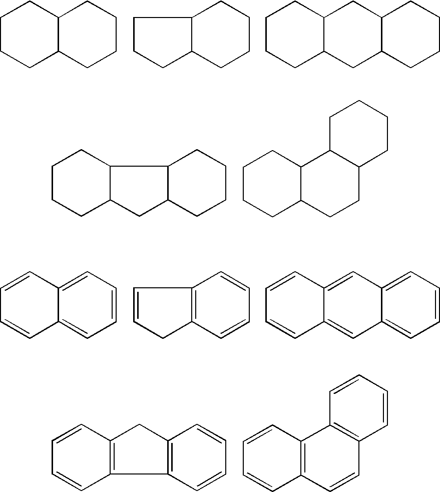

wherein said groups optionally comprise one to four heteroatoms selected from halogens, N, O and S.

In a second embodiment, the X chain comprises one or two carbon atoms which may be subtituted by an oxo group.

A preferred embodiment of the invention is directed to a method for treating or preventing a PPAR-γ mediated disease or condition comprising administration of a therapeutically effective amount of a compound the formula I, wherein said compound is

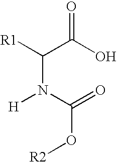

wherein R1 is selected from a linear or branched alkyl, alkenyl and alkynyl group comprising from 1 to 6 carbon atoms,

R2 is a polycyclic group selected from

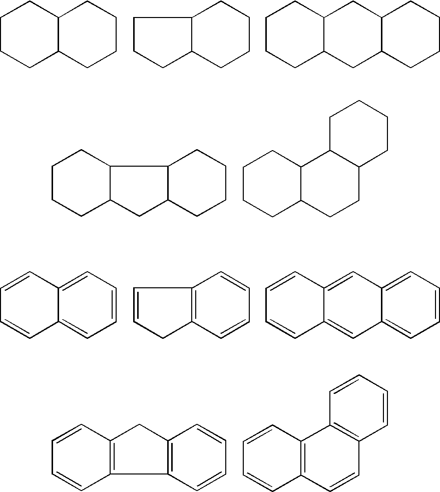

wherein said groups optionally comprise one to four heteroatoms selected from halogens, N, O and S.

Compounds that are more particularly suitable have the formula:

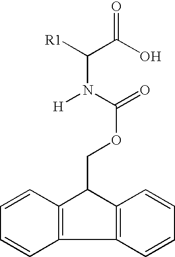

wherein R1 is selected from a linear or branched alkyl, alkenyl and alkynyl group [symbol] comprising from 1 to 6 carbon atoms and wherein the said tricyclic group optionally [symbol] comprises one to four heteroatoms selected from halogens, N, O and S. For example, a preferred compound is

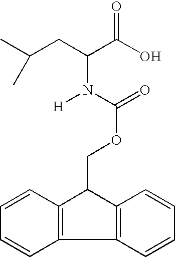

wherein the said tricyclic group optionally comprises one to four heteroatoms selected [symbol] from halogens, N, O and S; such as N-(9-fluroroenylmethyloxycarbonyl)-L-Leucine.

The method according to the invention is useful for treating or preventing anorexia, for [symbol] increasing or decreasing body weight, treating or preventing hyperlypidemia, for [symbol] increasing insulin sensitivity and for treating or preventing insulin resistance, as occurs [symbol] in diabetes.

Among the other diseases or conditions that can be treated or prevented with the [symbol] compounds described above, chronic inflammatory disorders such as inflammatory [symbol] bowel disease, ulcerative colitis, Crohn's disease, arthritis, notably rheumatoid [symbol] arthritis, polyarthritis and asthma are relevant.

The invention can also be reduced to practice for cancer, notably colon, prostate and [symbol] hematological cancer, as well as for atherosclerosis and skin disorders, notably [symbol] psoriasis.

We tested a number of the FMOC-aminoacid series for PPARγ-binding and activation. [symbol] Interestingly, whereas FMOC-L-tyrosine, which was structurally most similar to the L [symbol] tyrosine based PPARγ ligands (Cobb et al., 1998; Collins et al., 1998), was devoid of [symbol] PPARγ-activating properties, another member of the FMOC-aminoacid series, F-L-Leu [symbol] bound and activated PPARγ in a comprehensive set of in vitro and in vivo tests. [symbol] Evidence supporting FMOC-L-leucine as a stereoselective PPARγ agonist ligand is [symbol] provided by the following arguments:

1) F-L-Leu binds in vitro to PPARγ as evidenced by ESI-mass spectrometry (FIG. 5) [symbol] and protease protection assays (FIG. 4);

2) F-L-Leu enhanced co-activators recruitment to the PPARγ protein (FIG. 6);

3) F-L-Leu activates PPARγ in cotransfection studies (FIG. 3);

4) F-L-Leu induces adipocyte differentiation as judged by increased lipid [symbol] accumulation and the induction of adipocyte target genes, such as LPL and aP2 [symbol] (FIG. 7);

(5) F-L-Leu acts as a potent insulin-sensitizing agent in both diabetic and more [symbol] interestingly also in non-diabetic murine models (FIG. 8);

6) Finally, like the TZDs ((Jiang et al., 1998; Su et al., 1999), F-L-Leu also had [symbol] significant anti-inflammatory activities and could prevent inflammatory bowel [symbol] disease (FIG. 9). Since F-L-Leu is clearly structurally different from [symbol] thiazolidinediones and L-tyrosine based PPARγ ligands (Cobb et al., 1998; Collins [symbol] et al., 1998) and since F-L-Leu presents little or no structural analogies with the [symbol] partial agonists GW0072 (Oberfield et al., 1999) and L-764406 (Elbrecht et al., [symbol] 1999) and the antagonist BADGE (bisphenol A diglycidyl ether) (Wright et al., [symbol] 2000), F-L-Leu defines a chemically new class of PPARγ ligands.

Although F-L-Leu shares several functional characteristics with known PPAR [symbol] ligands, an important number of features distinguish F-L-Leu from these compounds [symbol] which will be addressed hereinafter.

F-L-Leu possesses an acidic function with the ability to liberate a proton, provided by [symbol] its carboxylic group. This is a feature shared by the natural ligand, PG J2, as well a [symbol] previously developed L-tyrosine based ligands. Such an acidic function is also present [symbol] in the TZD ring at the level of the nitrogen located between the two carbonyl groups. A [symbol] carboxylic group is also recovered in other PPARγ ligands such as GW0072, a weak [symbol] partial agonist which antagonizes adipocyte differentiation, but in which lateral side [symbol] chain substitution is approximately ten carbon atoms distant from the carboxylate [symbol] (Oberfield et al., 1999). This distance is in contrast with agonists such as the tyrosine derived ligands and F-L-Leu where a side-chain substitution occurs on the alpha-amino [symbol] position. The stereoselectivity of the activation of PPARγ with the FMOC L- and D [symbol] leucines (L- by far more potent than the D-enantiomer) confirms previous observation made on other ligands with an asymmetric carbon including 8-HETE (S>R) and a [symbol] alpha-trifluoroethoxy-propanoic acid derivative (S>R) (Rangwala et al., 1997; Young [symbol] et al., 1998; Yu et al., 1995).

Experimental evidence suggests that F-L-Leu would interact with the ligand binding site of PPARγ in a fashion distinct from both the TZDs and the tyrosine-based ligands Nuclear receptors generally only will dock one ligand molecule in their ligand binding pocket, which fits rather tightly around the ligand. Binding of both TZDs and tyrosine based PPARγ ligands follows this paradigm of “1 ligand/1 receptor” (Willson et al. 2000). The rather spacious ligand binding pocket of PPARγ, however, would not only allow the binding of large ligands, such as the tyrosine-based ligands, but eventually also allow binding of multiple ligand molecules to a single receptor. Our ESI-mass spectrometry data confirm that this is in fact the case with the F-L-Leu, where two molecules are shown to be bound to PPARγ ligand binding domain.

The capacity of PPARγ to bind two molecules of F-L-Leu could underly in fact some of the particular biological characteristics of this ligand. In fact, in transfection experiments F-L-Leu was two orders of magnitude less potent than the TZD rosiglitazone, although both compounds had similar maximal efficacy. The lower potency and the steep dose-response curve could therefore be explained by the fact that a higher molar ratio of ligand to receptor is required to change its configuration, finding consistent both with the results of our protease protection (FIG. 4) and cofactor interaction assays (FIG. 6).

Although, less potent in vitro, F-L-Leu compares rather favorably to TZDs, such a rosiglitazone, for anti-diabetic activity in vivo. Administration of F-L-Leu (1 mg/kg/day) to the diabetic db/db mice improved insulin sensitivity more dramatically than an equivalent dose of rosiglitazone. This could be deduced from the more robust reduction of the AUC in IPGTT for an almost equivalent reduction in fasting insulin levels. Furthermore, at a dose of 30 mg/kg/day F-L-Leu was able to significantly improve insulin sensitivity in normal animals, an effect never observed with glitazones. One caveat with this comparison between the in vivo efficacy of F-L-Leu relative to the TZDs, lies in the intraperitoneal route of drug administration used here. This is the only route described at present to be effective for F-L-Leu, but it is known to be suboptimal for TZDs, which are readily orally bioavailable. Despite this potential draw-back, the results obtained with F-L-Leu as a potential anti-diabetic drug remains however, remarkable. Moreover, F-L-Leu structure does not share a TZD ring, but offers a isosteric version of this chemical group via a carboxylic function (see FIG. 1), which is devoid of TZD-related side effects.

In summary, we describe F-L-Leu as a small synthetic PPARγ ligand. Unlike known PPARγ ligands, two molecules of F-L-Leu bind to a single PPARγ molecule, making its mode of receptor interaction novel and interesting. This unique way of receptor interaction, underlies some of the particular pharmacological properties of F-L-Leu. In general, F-L-Leu exerts similar biological activities as the known groups of PPAR [symbol] agonists, with a distinct pharmacology, characterized by a lower potency, but similar maximal efficacy. This novel synthetic molecule represents hence a ne [symbol] pharmacophore, which can be optimized according to routine procedures, for modulation of PPARγ biological activity.

### FIGURE LEGENDS

FIG. 1: Schematic representation of PPARγ ligand structures. The different routes followed for the design are indicated.

A. anti-diabetic glitazones

B. L-tyrosine based PPARγ ligands

C. FMOC amino acids

FIG. 2: Modulation of transcriptional activity of PPARγ2 by FMOC-amino-acid in Hep G2 cells. Hep G2 cells were co-transfected with an expression vector for PPARγ2 (0.1 μg/well), pGL3-(J_{wt})₃TKLuc reporter construct (0.5 μg/well), and pCMV-βGal (0.5 μg/well), as a control of transfection efficiency (0.5 μg/well). The were then grown during 24 h in the presence or absence of indicated compound Activation is expressed as relative luciferase activity/β-galactosidase activity. Each point was performed in triplicate. This figure is representative of three independent experiments.

FIG. 3: F-L-Leu enhances transcriptional activity of PPARγ2 in different cell lines. RK13 cells (A and D), CV1 cells (B) or Hep G2 cells (C) were co-transfected with an expression vector for PPARγ2 (0.1 μg/well), pGL3-(J_{wt})₃TKLuc reported construct (0.5 μg/well), and pCMV-βGal (0.5 μg/well), as a control of transfection efficiency (0.5 μg/well). They were then grown during 24 h in the presence or absence of indicated compound. Activation is expressed as relative luciferase activity/β [symbol] galactosidase activity. Each point was performed in triplicate, and each figure i [symbol] representative of four independent experiments.

FIG. 4: F-L-Leu ligand alters the conformation of PPARγ. ³⁵S-PPARγ wa [symbol] synthesized in vitro in a coupled transcription/translation system. Labeled PPARγ wa [symbol] subsequently incubated with DMSO (0.1%), rosiglitazone (10⁻⁴M) or F-L-Leu (10⁻⁴M [symbol] followed by incubation with distilled water or increasing concentrations of trypsin. Digestion products were analyzed by SDS-PAGE followed by autoradiography. The migration of intact PPARγ is indicated and the asterisk indicates the 25-kDa resistant fragment of PPARγ.

FIG. 5: Two molecules of F-L-Leu bind to a single PPARγ molecule. ESI-mass spectrometry analysis.

FIG. 6: F-L-Leu enhances the interaction of PPARγ with p300. The purified his-tagPPARγ2_{DE203-477} protein was incubated with purified p300Nt-GST protein and glutathione-Q-Sepharose beads in presence of DMSO (0.1%), rosiglitazone (10⁻⁴ M) or F-L-Leu (10⁻³ M). The beads were then washed and the samples separated on SDS-PAGE and blotted. The blot was developed with anti-histidine antibodies.

FIG. 7: F-L-Leu enhances adipocyte differentiation. Confluent 3T3-L1 cells were incubated with 2 μM insulin, 1 μM dexamethasone, and 0.25 mM isobuthyl methyl xanthine for two days. Then, the cells were incubated in presence of DMSO (0.1%), F-L-Leu (10⁻⁵ M) or rosiglitazone (10⁻⁷ M) for 4 days. A: RNA was isolated from 3T3-L1 cells after different times of differentiation induction. Blots were hybridized with 36B4 (to control for RNA loading); LPL or aP2 cDNAs. B: Cells were stained with Oil Red O after 6 days. LPL: lipoprotein lipase.

FIG. 8: F-L-Leu improves insulin sensitivity in C57BL/6j and db/db mice. Intraperitoneal glucose tolerance test (IPGTT) in C57BL/6j (A) or db/db (B) mice 10 to 12 weeks old (n=8). Diamonds correspond to DMSO-treated mice; squares to F-L-Leu-treated mice at the concentration of 10 mg/kg/day and triangles to F-L-Leu-treated mice at the concentration of 30 mg/kg/day (for C57BL/6j mice, A) or rosiglitazone-treated mice at the concentration of 10 mg/kg/day (for db/db mice, B). Insulinemia (C) and body weights (D) of db/db mice treated with DMSO, F-L-Leu (10 mg/kg/day) or rosiglitazone (10 mg/kg/day).

FIG. 9: F-L-Leu protects against colon inflammation in TNBS-treated Balb/c Mice. A: Ameho histologic scores (left panel) and survival rate (right panel) in TNBS-treated mice injected either with DMSO or F-L-Leu (50 mg/kg/day). B: TNFα and IL-1β mRNA levels in the colon of TNBS-treated mice injected with DMSO or F-L-Leu (50 mg/kg/day). Results are expressed as mean±SEM.

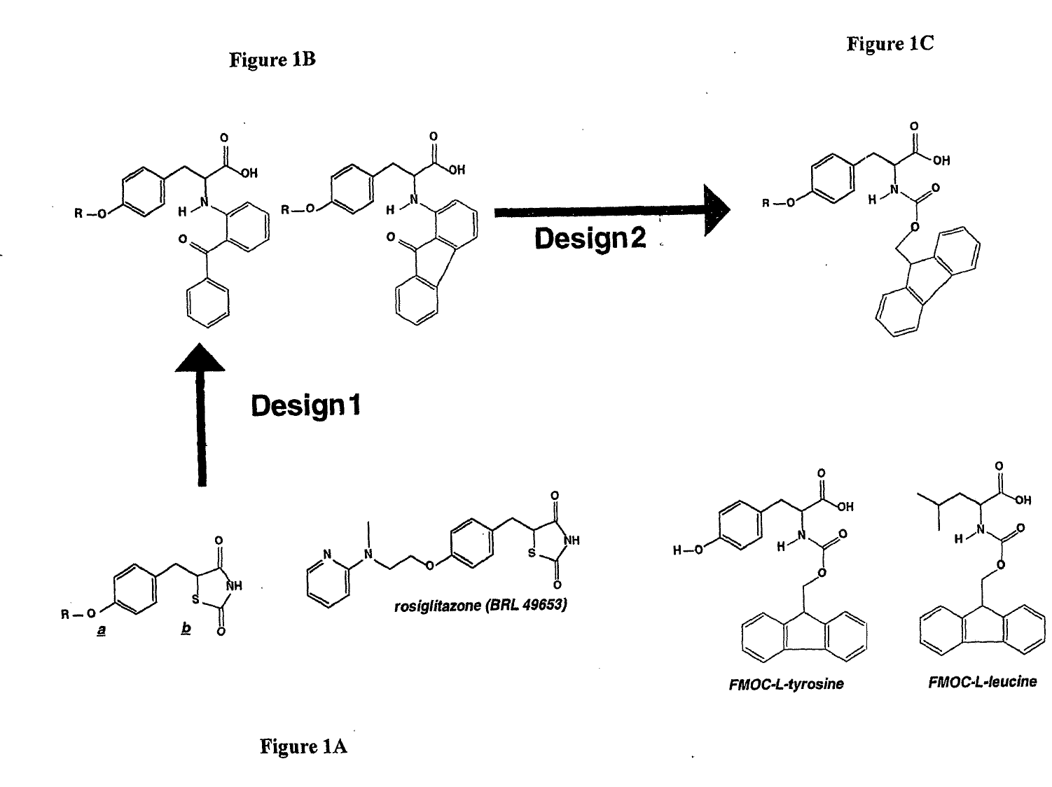

The following materials and methods were used to perform the examples below.

Materials and Methods

FMOC-derivatives were acquired at Sen Chemicals (Dielsdorf, Switzerland). Rosiglitazone and pioglitazone were kind gifts of Dr. R. Heyman (Ligand Pharmaceuticals, San Diego, Calif.). The antibodies directed against the AB domain of PPARγ were produced in our laboratory (Fajas et al., 1997). The protease inhibitor cocktail was purchased at ICN (Orsay, France).

Cell Culture and Transient Transfection Assays

The CV1, RK-13, and Hep G2 cell lines were obtained from ATCC (Rockville, Md.). Cells were maintained in Dulbecco's modified Eagle's minimal essential medium (DMEM) supplemented with 10% fetal calf serum (FCS), L-glutamine, and antibiotics. Transfections with chloramphenicol acetyltransferase (CAT) or luciferase (luc.) reporter constructs were carried out exactly as described previously (Schoonjans et al., 1996). The pGL3-(J_{wt})₃TKLuc and the pGL3-(J_{wt})₃TKCAT reporter constructs contain both three tandem repeats of the J site of the apolipoprotein A-II promoter cloned upstream of the herpes simplex virus thymidine kinase (TK) promoter and the luciferase or the CAT reporter genes respectively (Vu-Dac et al., 1995). The following expression vectors were used; pSG5-hPPARγ2, a construct containing the entire cDNA of the human PPARγ2 (hPPARγ2) (Fajas et al., 1997); pSG5-mPPARα (Isseman et al., 1993); and pCMV-βGal, as a control of transfection efficiency.

Production of Proteins and Mass Spectrometry

The p300Nt-GST, fusion protein was generated by cloning the N-terminal part of the p300 protein (a.a. 2 to 516) downstream of the glutathione-S-transferase (GST) protein in the pGex-T1 vector (Pharmacia, Orsay, France). The fusion proteins were then expressed in Escherichia coli and purified on a glutathione affinity matrix (Pharmacia). Human PPARγ (aa. 203 to 477 of PPARγ) was subcloned into the pET15b (Novagen, Madison, Wis.) expression vector. The his-tagPPARγ2_{DE203-477} proteins were produced as follow. The protein was purified using a metal chelate affinity column with an affinity column Co²⁺ coupled agarose (High Trap chelatin, Pharmacia). The protein was eluted with 20 mM Tris-HCl, 500 M NaCl, 130 mM imidazole and 1-2 propanediol 2.5% (pH 8.5). A second purification step was made by gel filtration (Superdex 200 16/60, Pharmacia). The protein was eluted with 20 mM Tris-HCl, 100 mM NaCl, 5 mM DTT and 1-2 propanediol 2,5% (pH 8.5). Liquid chromatography-electrospray ionization (ESI)-mass spectrometry analysis was performed as previously described (Rogniaux et al., 1999).

Protease Protection and Pull-Down Experiments

Protease Protection Experiments.

The pSG5-hPPARγ2 plasmid was used to synthesize [symbol] ³⁵S-radiolabeled PPARγ in a coupled transcription/translation system according to the protocol of the manufacturer (Promega, Madison, Wis.). The transcription/translation reactions were subsequently aliquoted into 22.5 μl and 2,5 μl of phosphate buffered saline +/− compound were added. The mixture was separated into 4.5 μl aliquots and 0.5 μl of distilled water or distilled water-solubilized trypsin were added. The protease digestion were allowed to proceed for 10 min at 25° C. and terminated by the addition denaturing loading buffer. After separation of the digestion products in a gel SDS [symbol] PAGE 12% acrylamide, the gel was fixed in 10% acetic acid (v/v) 30% ethanol (v/v) [symbol] for 30 min, treated in Amplify™ (Amersham, Orsay, France) and dried. The radiolabeled digestion products were visualized by autoradiography.

Pull-Down Experiments.

The purified his-tagPPARγ DE proteins were incubated 1 ho [symbol] at 22° C. in pull-down buffer (phosphate-buffered saline 1×, Glycerol 10%, NP40 0.5% [symbol] with either GST or p300Nt-GST fusion protein, glutathione-Q sepharose beads, and F [symbol] L-Leu (10⁻³M) or rosiglitazone (10⁻⁴M) when necessary. The beads were then washed [symbol] 4 times in pull-down buffer and boiled in 2× sample buffer. The samples wer [symbol] separated by 12% acrylamide SDS-PAGE and transferred to nitrocellulose membrane [symbol] Blots were developed with antibodies directed against polyhistidine aminoaci [symbol] sequences.

Adipocyte Differentiation

3T3-L1 cells (ATCC, Rockville, Md.) were grown to confluence in medium [symbol] (Dulbecco's modified Eagle's Medium with 10% fetal calf serum, 100 units/n [symbol] penicillin, and 100 μg/ml streptomycin). Confluent cells were incubated in medium [symbol] containing 2 μM insulin, 1 μM dexamethasone, and 0.25 mM isobuthyl meth [symbol] xanthine for two days. Then, the cells were incubated in medium A in presence [symbol] absence of PPARγ y agonist for 4 days, changing the medium every 2 day [symbol] Adipogenesis was evaluated by analysis of the expression of adipocyte-specif [symbol] markers and by staining of lipids with Oil Red O (Chawla and Lazar, 1994).

RNA Preparation and Analysis

RNA was isolated from 3T3-L1 cells by the acid guanidinium [symbol] thiocyanate/phenol/chloroform method (Chomczynski and Sacchi, 1987). Northern blot analysis of total cellular RNA was performed as described (Auwerx et al., 1989) [symbol] Lipoprotein lipase (LPL), aP2 and 36B4 were used as probes (Graves et al., 1992 [symbol] Laborda, 1991; Lefebvre et al., 1997). For RT-competitive PCR, total RNA (5-10 μg [symbol] was reverse transcribed into complementary DNA (cDNA) (Desreumaux et al., 1999 [symbol] Fajas et al., 1997). The RT reaction mixture was amplified by PCR using sense an [symbol] antisense primers specific for β-actin, TNFα and IL-1β. The samples were subjected t [symbol] 40 PCR cycles, consisting of denaturation for 1 min at 94° C., primer annealing for [symbol] min at 52-58° C., and primer extension for 1.5 min at 72° C. using a Gene Amp PCR [symbol] System 9700 (Perkin-Elner Corporation, Foster City, Calif.). The quantity of mRNA was expressed as the number of TNFα or IL-1β cDNA per β-actin cDNA molecules.

Animal Experiments, Glucose Metabolism and Inflammation

All mice were maintained in a temperature-controlled (25° C.) facility with a strict 12 h [symbol] light/dark cycle and were given free access to food (standard mice chow; DO4, UAR [symbol] France) and water. Animals received F-L-Leu or rosiglitazone by intraperitones [symbol] injection.

C57B1/6J and db/db mice (8 per group) were obtained through the Janvier laboratorie [symbol] (Laval-Le Genest, France). Intraperitoneal glucose tolerance tests (IPGTT) wer [symbol] performed as described (Kaku et al., 1988). Briefly, mice were fasted overnight (18 [symbol] and injected intraperitonealy (i.p.) with 25% glucose in sterile saline (0.9% NaCl) [symbol] a dose of 2 g glucose/kg body weight. Blood was subsequently collected from the ta [symbol] for glucose quantification with the Maxi Kit Glucometer 4 (Bayer Diagnostic, Puteaux [symbol] France) prior to and at indicated times after injection. Blood for insulin measurement [symbol] was collected in fasting mice from the retroorbital sinus plexus under chloroform anesthesia. Plasma was separated and insulin measured using a radio immunoassay kit (Cis bio international, Gif-sur-Yvette, France).

Male Balb/c mice (8 per group) were used for the colitis studies (Jackson laboratories, Bar Harbor, Me.). Colitis was induced by administration of 40 μl of a solution of TNBS (150 mg/kg, Fluka, Saint Quentin Fallavier, France) dissolved in NaCl 0.9% and mixed with an equal volume of ethanol (50% ethanol). This solution was administered intrarectally via a 3.5 F catheter (Ref EO 3416-1, Biotrol, Chelles, France) inserted 4 cm proximal to the anus in anesthesized mice [Xylasine (50 mg/kg of Rompun® 2%, Bayer Pharma, Puteaux, France) and Ketamine (50 mg/kg of Imalgene® 1000, Rhône Mérieux, France)]. Animals were sacrified by cervical dislocation under ether anesthesia two days after TNBS administration. The colon was quickly removed, opened, washed. A 2 cm colonic specimen located precisely 2 cm above the anal canal was dissected systematically in 4 parts. One part was fixed overnight in 4% paraformaldehyde acid at 4° C., dehydrated in alcohol and embedded in paraffin. Sections (5 μm) were then deparaffined with xylene and rehydrated by ethanol treatment. Stained sections with haematoxylin-eosin were examined blindly by a pathologist and scored according to the Ameho criteria (Ameho et al., 1997). The other parts of the colon were used for RNA isolation for the quantification of TNFα and IL1β mRNA expression.

Statistical Analysis

Values were reported as mean +/− standard deviation. Statistical differences were determined by the Mann-Whitney U test. P<0.05 was accepted as statistically significant.

### EXAMPLE 1

### FMOC-L-Leucine Activates PPARγ in Cell Transfection Experiments

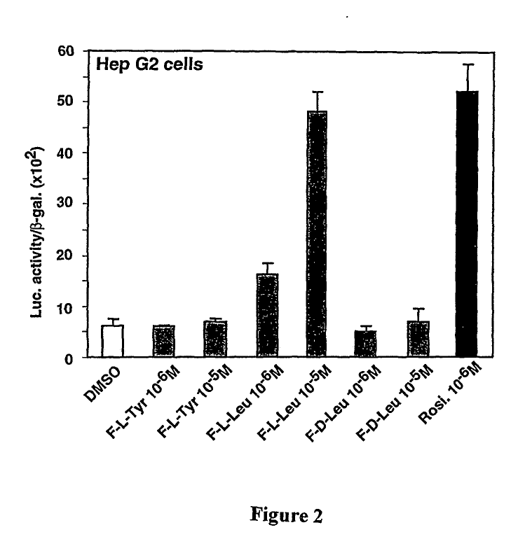

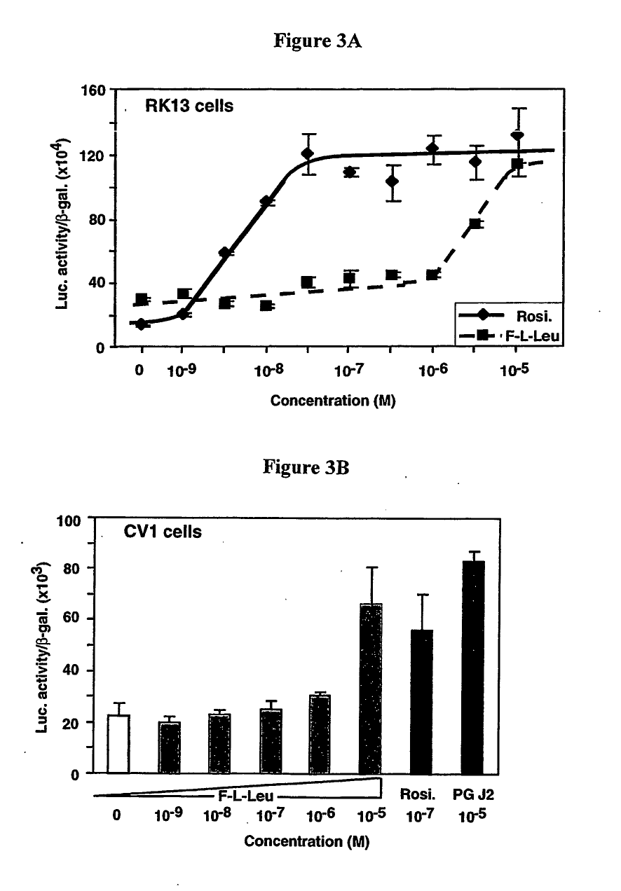

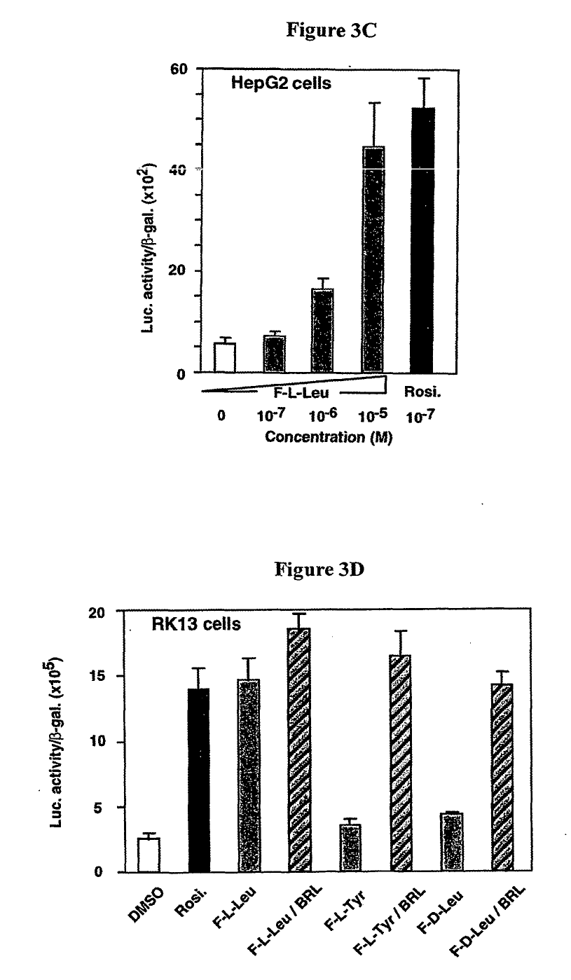

Various FMOC derivatives of unsubstituted (L-tyrosine, D-leucine, and L-leucine) aminoacids were tested and compared to rosiglitazone or pioglitazone (as positive internal controls) for their ability to activate PPARγ in transient transfection experiments in HepG2 cells using the pSG5-hPPARγ2 expression and J₃TKpGl₃ reporter plasmids. In contrast to L-tyrosine PPARγ ligands (Cobb et al., 1998; Collins et al., 1998), the FMOC substituted L-tyrosine derivative did not activate PPARγ. Significant PPARγ activity could, however, be detected for F-L-leu at the concentration of 10⁻⁵ M (FIG. 2). In contrast, no significant PPARγ activation was detected with the FMOC-D-leucine derivative, demonstrating that PPARγ activating properties of F-L-leu were stereoselective. Additional transfection experiments with F-L-Leu were performed on different cell lines (RK13, CV1 and HepG2 cells) (FIG. 3 A, B and C). In the rabbit kidney RK13 cells, we found that rosiglitazone has an optimal activity between 10⁻⁸ to 10⁻⁷ M. For F-L-Leu, PPARγ activation occurred at concentrations of 10⁻⁵ M (FIG. 3A). Consistent with previous results, F-L-Leu concentrations of 10⁻⁵ M were also required for optimal PPARγ activation in simian renal cells CV1 (FIG. 3B), and in human HepG2 cells (FIG. 3C). The optimal concentration for PPARγ activation by F-L-Leu was similar to that of PG J2 and 100-fold higher than the concentration of rosiglitazone (FIG. 3C) or pioglitazone (data not shown) necessary to reach the same efficacy.

Finally, we tested whether FMOC-amino acid derivatives synergized or antagonized rosiglitazone activation of PPARγ in RK13 cells (FIG. 3D). No significant modification of PPARγ activity was observed when we added either F-L-Leu, FMOC L-tyrosine or FMOC D-leucine (10⁻⁵ M) to a saturating concentration of rosiglitazone.

These results furthermore confirmed (see FIG. 3A) that we reached maximal PPARγ [symbol] activation using rosiglitazone and F-L-Leu at the concentration of 10⁻⁷M and 10⁻⁵M [symbol] respectively.

### EXAMPLE 2

### FMOC L-Leucine Changes PPARγ Conformation

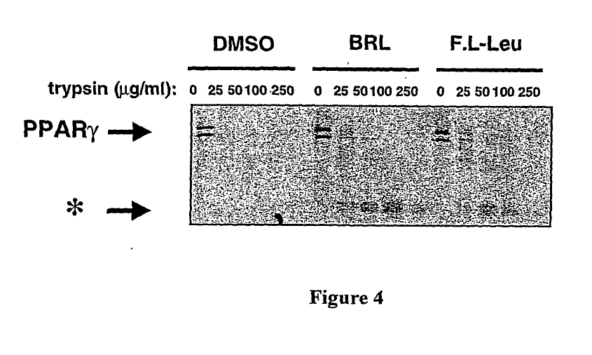

Thiazolidinediones can induce an alteration in the conformation of PPARγ, as assesse [symbol] by generation of protease-resistant bands following partial trypsin digestion o [symbol] recombinant receptor (Berger et al., 1999; Elbrecht et al., 1999). Upon incubation o [symbol] rosiglitazone with PPARγ, a fragment of approximately 25 kDa is protected fro [symbol] trypsin digestion whereas no protection is detected when PPARγ is incubated with [symbol] DMSO vehicle (FIG. 4). Interestingly, F-L-Leu produced a protease protection [symbol] pattern similarly to rosiglitazone, demonstrating that F-L-Leu altered [symbol] PPARγ conformation (FIG. 4).

### EXAMPLE 3

### Two Molecules of FMOC-L-Leucine Interact with PPARγ

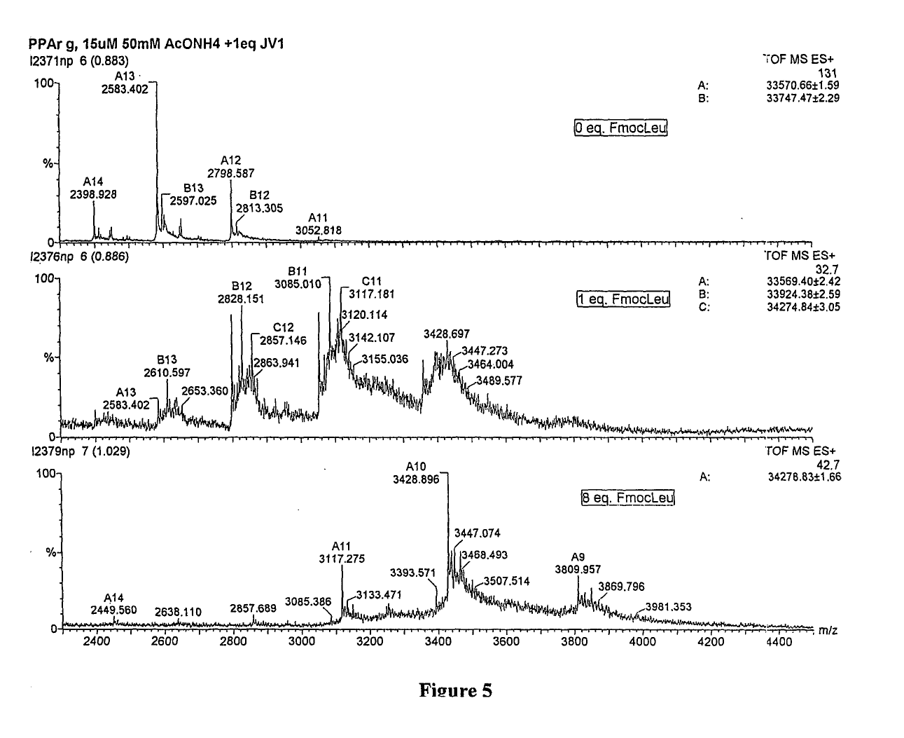

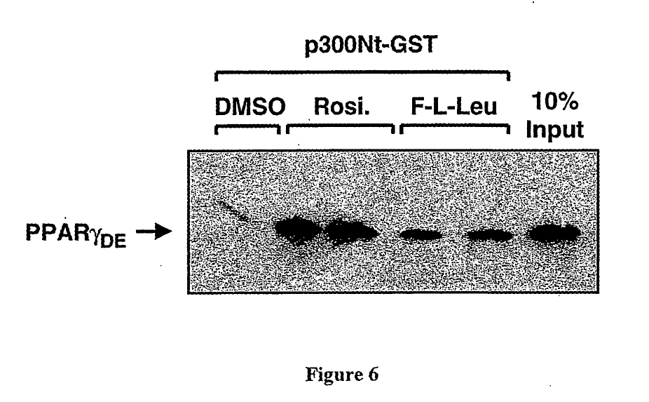

Electrospray ionization (ESI) mass spectrometry of hPPARγ LBD (amino acid 203 t [symbol] 477) was used to identify the specific binding of F-L-Leu with PPARγ (FIG. 5). Th [symbol] purified fragment of PPARγ LBD was incubated with vehicle alone or either 1 or [symbol] equivalents of F-L-Leu per equivalent of PPARγ. The mass of the receptor wa [symbol] determined after incubation by ESI-mass spectrometry. At 1 equivalent of F-L-Leu p [symbol] equivalent of PPARγ, we could distinguish three populations of PPARγ correspondin [symbol] to: 1/ unliganded PPARγ; 2/ a complex formed by 1 PPARγ LBD molecule and 1 F-L [symbol] Leu molecule; and 3/ a complex formed by 1 PPARγ LBD molecule and 2 F-L-Le [symbol] molecules. Interestingly, when we increased the F-L-Leu concentration (8 equivalents of F-L-Leu per 1 equivalent of PPARγ), we detected only the complex correspondin [symbol] to the PPARγ LBD bound with 2 F-L-Leu molecules. These results indicate that two molecules of F-L-Leu interact with one molecule of the PPARγ in a highly specific manner.

### EXAMPLE 4

### FMOC-L-Leucine Enhances PPARγ/p300 Interaction

PPARγ has been previously reported to interact with the cofactor p300. The overall molecular PPARγ/p300 interaction was the resultant of a ligand-independent binding of p300 to PPARγs' ABC domain and a ligand-dependent interaction of p300 with the PPARγ DE domains (Gelman et al., 1999). Hence the purified PPARγ DE protein represents a tool to study the efficacy of PPARγ ligand binding properties in view of its' ability to recruit p300 upon ligand binding. Compared to the DMSO control, both rosiglitazone and F-L-Leu effectively induced the formation of PPARγ DE/p300Nt-GST complexes. This confirms that the F-L-Leu is a PPARγ ligand and that its binding to the PPARγ DE domain is capable of inducing conformational changes required for association with p300. The potency of the F-L-Leu compound was in this assay 2- to 3-fold lower than that of rosiglitazone.

### EXAMPLE 5

### FMOC-L-Leucine Induces Adipocyte Differentiation

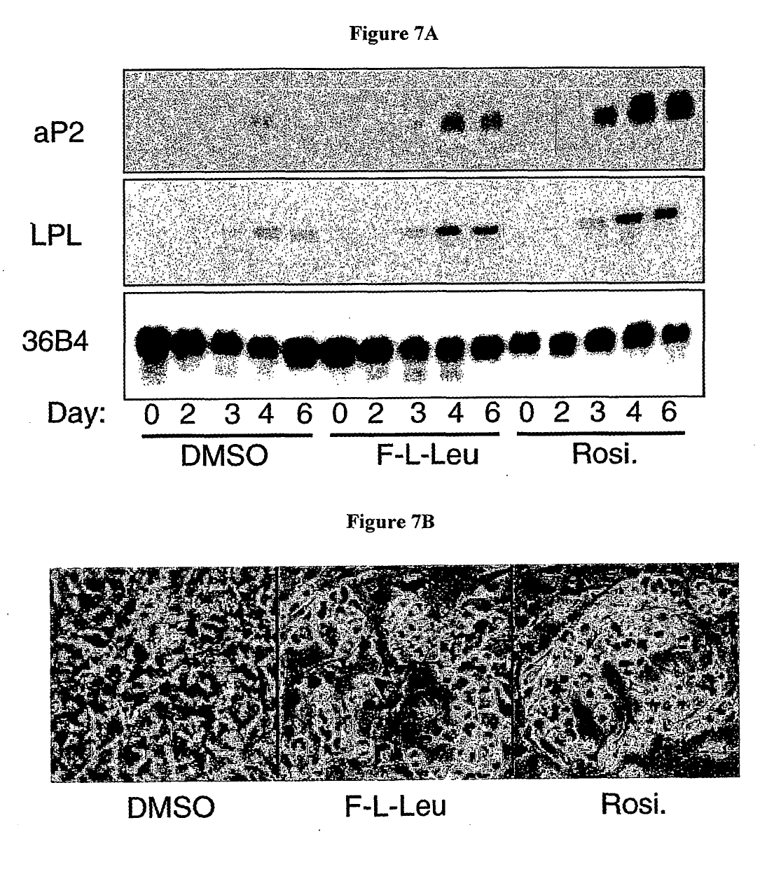

The ability of F-L-Leu and rosiglitazone to stimulate adipocyte differentiation o [symbol] murine pre-adipocyte 3T3-L1 cells were next compared. Adipogenesis was monitored by analysis of lipoprotein lipase (LPL) and aP2 mRNA levels as markers of adipocyte differentiation and by studying morphological changes associated with the differentiation process. F-L-Leu at the concentration of 10⁻⁵ M significantly stimulated both LPL and aP2 mRNA levels to an extent close to that seen in cells incubated with rosiglitazone at the concentration of 10⁻⁷ M (FIG. 7A). Staining of 3T3-L1 cells with Oil Red O, as a marker for neutral lipid accumulation, was performed after a 6 days incubation of cells with either DMSO, or the two PPARγ ligands F-L-leu or rosiglitazone (FIG. 7B). The two drugs were again capable of inducing neutral lipid accumulation. Hence, like rosiglitazone, F-L-Leu was an adipogenic drug in 3T3-L1 cells.

### EXAMPLE 6

### FMOC-L-Leucine Improves Insulin Ensitivity In Vivo

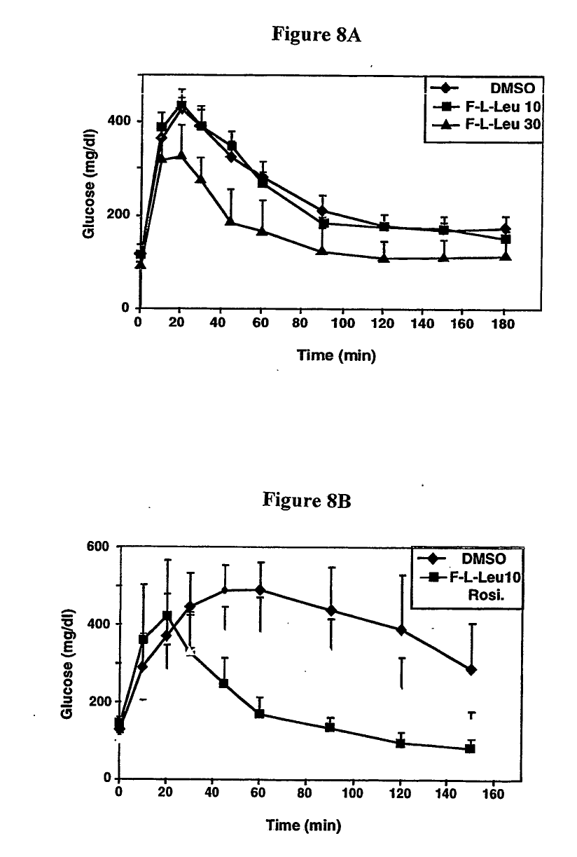

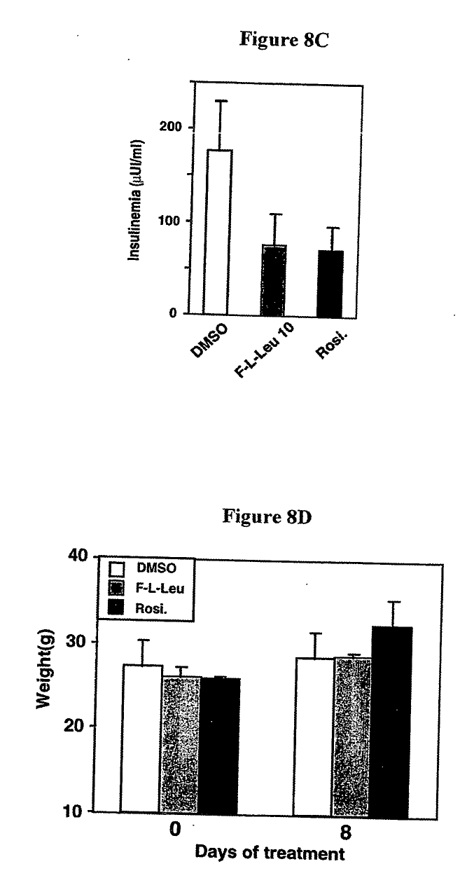

To assess whether F-L-Leu could improve insulin sensitivity, we compared the glucose tolerance in C57BL/6j mice treated with F-L-Leu relative to that observed in control animals which received only the vehicle, DMSO (FIG. 8A). Mice were treated with 2 different doses of F-L-Leu (10 and 30 mg/kg/days) during 7 days and then IPGTT was performed. Intra-peritonealy administrated glucose was cleared in a comparable rate in mice receiving vehicle or F-L-Leu at 10 mg/kg/day. In mice treated with F-L-Leu at 30 mg/kg/day, the maximum glucose levels increased only to 320 mg/dl whereas the glucose levels climbed to 440 mg/dl after glucose injection for both 10 mg/kg/day F-L-Leu and the control group. Furthermore, the area under the curve was significantly lower in mice treated with F-L-Leu at 30 mg/kg/day relative to either control mice or mice receiving F-L-Leu at lower dose.

We next compared glucose tolerance in db/db mice treated with DMSO, F-L-Leu (10 mg/kg/day) or rosiglitazone (10 mg/kg/day) during 7 days. In control mice (DMSO group), glycemia rapidly increased after glucose loading, reaching a maximum of 500 mg/dl between 45 to 60 min after injection, before slowly decreasing. In rosiglitazone-treated mice, glucose loading was better “tolerated” than in control animals with a reduction in the maximal glycemia (350 mg/dl), and a more rapid recovery of these supranormal values. F-L-Leu-treated animals showed the best glucose tolerance test, with a maximal glucose level (420 mg/dl) 20 min after injection and an immediate and fast subsequent decrease to normal (100 mg/dl) values within 120 min. Furthermore, 7 days treatment of animals with F-L-Leu and rosiglitazone resulted in a dose-dependent lowering of fasting serum insulin levels (mean values of 70 μUI/mL for db/db mice treated with either F-L-Leu or rosiglitazone versus 180 μUI/mL for the DMSO group) (FIG. 8C). These data clearly show that F-L-Leu improves insulin sensitivity in both diabetic and normal mice. Interestingly, whereas rosiglitazone had a tendency to increase body weight of mice, no difference in body weight was seen in mice treated with F-L-Leu during 8 days when compared to control mice (FIG. 8D). In addition, we observed a tendency to diminution of the liver weight for F-L-Leu-treated mice relative to control or rosiglitazone-treated mice (data not shown).

### EXAMPLE 7

### FMOC-L-Leucine Protects Against Colitis

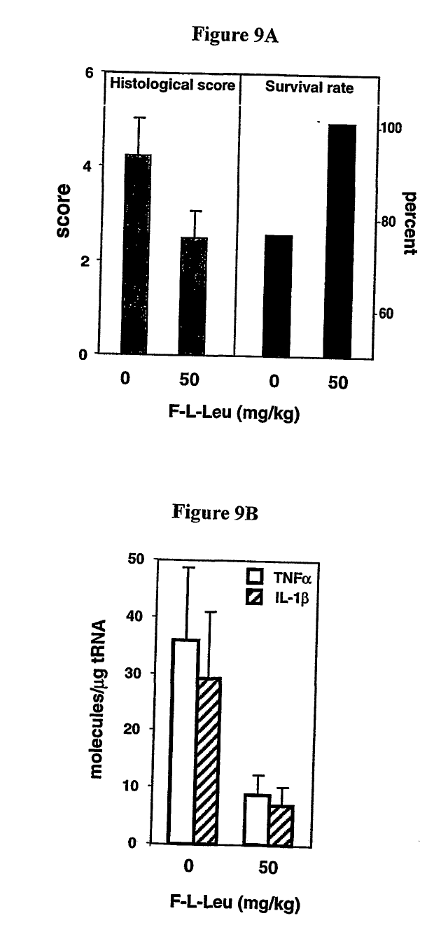

Intrarectal administration of TNBS has been shown to induce rapidly and reproducibly a colitis in mice as a result of covalent binding of TNP residues to autologuous host proteins leading to a mucosal infiltration by polynuclear cells, the production of TNFα, and the activation of NFkB (Allgayer et al., 1989; Stenson et al., 1992; Su et al., 1999).We determined the survival rate and scored the colon damage as well as the production of cytokines two days after intra-rectal TNBS administration in control animals or animals which were treated 4 days with F-L-Leu at 50 mg/kg/day (FIG. 9). Interestingly, 100% of F-L-Leu-treated mice survived colon inflammation whereas only 76% of control mice were alive after induction of inflammation. Administration of F-L-Leu furthermore reduced significantly the histologic score indicating that F-L-Leu reduces ulceration, erosion and necrosis induced by inflammation. Finally, F-L-Leu administration resulted in a significant decrease in the mRNA levels expression of the pro-inflammatory cytokines, TNFα and IL-1β suggesting that, like with rosiglitazone, PPARγ activation by F-L-Leu protects against colon inflammation by inhibition of the TNFα signaling pathway.

References

Allgayer, H., Deschryver, K. and Stenson, W. F. (1989) Treatment with 16,16′-dimethyl prostaglandin E2 before and after induction of colitis with trinitrobenzenesulfonic acid in rats decreases inflammation. Gastroenterology, 96, 1290-1300.

Ameho, C. K., Adjei, A. A., Harrison, E. K., Takeshita, K., Morioka, T., Arakaki, Y., Ito, E., Suzuki, I., Kulkami, A. D., Kawajiri, A. and Yamamoto, S. (1997) Prophylactic effect of dietary glutamine supplementation on interleukin 8 and tumour necrosis factor alpha production in trinitrobenzene sulphonic acid induced colitis. Gut, 41, 487-493.

Auwerx, J., Chait, A., Wolfbauer, G. and Deeb, S. (1989) Involvement of second messengers in regulation of the low-density lipoprotein receptor gene. Mol. Cell. Biol., 9, 2298-2302.

Berger, J., Bailey, P., Biswas, C., Cullinan, C. A., Doebber, T. W., Hayes, N. S., Saperstein, R., Smith, R. G. and Leibowitz, M. D. (1996) Thiazolidinediones produce a conformational change in peroxisomal proliferator-activated receptor-γ: binding and activation correlate with antidiabetic actions in db/db mice. Endocrinology, 137, 4189-4195.

Berger, J., Leibowitz, M. D., Doebber, T. W., Elbrecht, A., Zhang, B., Zhou, G., Biswas, C., Cullinan, C. A., Hayes, N. S., Li, Y., Tanen, M., Ventre, J., Wu, M. S., Berger, G. D., Mosley, R., Marquis, R., Santini, C., Sahoo, S. P., Tolman, R. L., Smith, R. G. and Moller, D. E. (1999) Novel peroxisome proliferator activated receptor (PPAR) γ and PPARδ ligands produce disinct biological effects. J. Biol. Chem., 274, 6718-6725.

Burch, R. M., Weitzberg, M., Blok, N., Muhihauser, R., Martin, D., Farmer, S. G., Bator, J. M., Connor, J. R., Green, M., Ko, C. and et al. (1991) N-(fluorenyl-9-methoxycarbonyl) amino acids, a class of antiinflammatory agents with a different mechanism of action [published erratum appears in Proc Natl Acad Sci USA March 15, 1991; 88(6):2612]. Proc. Natl. Acad. Sci. USA, 88, 355-359.

Chawla, A. and Lazar, M. A. (1994) Peroxisome proliferator and retinoid signaling pathways co-regulate preadipocyte phenotype and survival. Proc. Natl. Acad. Sci. USA, 91, 1786-1790.

Chomczynski, P. and Sacchi, N. (1987) Single step method for RNA isolation by acid guanidinium-thiocyanate-phenol-chloroform extraction. Anal. Biochein., 162, 156-159.

Cobb, J. E., Blanchard, S. G., Boswell, E. G., Brown, K. K., Charifson, P. S., Cooper, J. P., Collins, J. L., Dezube, M., Henke, B. R., Hull-Ryde, E. A., Lake, D. H., Lenhard, J. M., Oliver, W., Jr., Oplinger, J., Pentti, M., Parks, D. J., Plunket, K. D. and Tong, W. Q. (1998) N-(2-Benzoylphenyl)-L-tyrosine PPARgamma agonists. 3. Structure-activity relationship and optimization of the N-aryl substituent. J. Med. Chem, 41, 5055-5069.

Collins, J. L., Blanchard, S. G., Boswell, G. E., Charifson, P. S., Cobb, J. E., Henke, B. R., Hull-Ryde, E. A., Kazmierski, W. M., Lake, D. H., Leesnitzer, L. M., Lehmann, J., Lenhard, J. M., Orband-Miller, L. A., Gray-Nunez, Y., Parks, D. J., Plunkett, K. D. and Tong, W. Q. (1998) N-(2-Benzoylphenyl)-L-tyrosine PPARgamma agonists. 2. Structure-activity relationship and optimization of the phenyl alkyl ether moiety. J Med Chem, 41, 5037-5054.

Desreumaux, P., Ernst, O., Geboes, K., Gambiez, L., Berrebi, D., Muller-Alouf, H., Hafraoui, S., Emilie, D., Ectors, N., Peuchmaur, M., Cortot, A., Capron, M., Auwerx, J. and Colombel, J. F. (1999) Inflammatory alterations in mesenteric adipose tissue in Crohn's disease. Gastroenterology, 117, 73-81.

Desvergne, B. and Wahli, W. (1999) Peroxisome proliferator-activated receptors: nuclear control of metabolism. Endocr. Rev., 20, 649-688.

Elbrecht, A., Chen, Y., Adams, A., Berger, J., Griffin, P., Klatt, T., Zhang, B., Menke, J., Zhou, G., Smith, R. G. and Moller, D. E. (1999) L-764406 is a partial agonist of human peroxisome proliferator-activated receptor gamma. The role of Cys313 in ligand binding. J Biol Chem, 274, 7913-7922.

Fajas, L., Auboeuf, D., Raspe, E., Schoonjans, K., Lefebvre, A. M., Saladin, R., Najib, J., Laville, M., Fruchart, J.C., Deeb, S., Vidal-Puig, A., Flier, J., Briggs, M. R., Staels, B., Vidal, H. and Auwerx, J. (1997) Organization, promoter analysis and expression of the hunan PPARγ gene. J. Biol. Chem., 272, 18779-18789.

Forman, B. M., Tontonoz, P., Chen, J., Brun, R. P., Spiegelman, B. M. and Evans, R. M. (1995) 15-Deoxy-Δ12,14 prostaglandin J2 is a ligand for the adipocyte determination factor PPARγ. Cell, 83, 803-812.

Gelman, L., Zhou, G., Fajas, L., Raspe, E., Fruchart, J. C. and Auwerx, J. (1999) p300 interacts with the N- and C-terminal part of PPARγ2 in a ligand-independent and -dependent manner respectively. J. Biol. Chem., 274, 7681-7688.

Graves, R. A., Tontonoz, P. and Spiegelman, B. M. (1992) Analysis of a tissue-specific enhancer: ARF6 regulates adipogenic gene expression. Mol. Cell. Biol, 12, 1202-1208.

Isseman, I., Prince, R. A., Tugwood, J. D. and Green, S. (1993) The peroxisome proliferator-activated receptor: retinoic X receptor heterodimer is activated by fatty acids and fibrate hypolipidaemic drugs. Journal of Molecular Endocrinology, 11, 3747.

Jiang, C., Ting, A. T. and Seed, B. (1998) PPARγ agonists inhibit production of monocyte inflammatory cytokines. Nature, 391, 82-86.

Kaku, K., Fiedorek, F. T., Province, M. and Permutt, M. A. (1988) Genetic Analysis of Glucose Tolerance in Inbred Mouse Stains. Diabetes, 37, 707-713.

Kliewer, S. A., Lenhard, J. M., Willson, T. M., Patel, I., Morris, D. C. and Lehman, J. M. (1995) A prostaglandin J2 metabolite binds peroxisome proliferator-activated receptor γ and promotes adipocyte differentiation. Cell, 83, 813-819.

Laborda, J. (1991) 36B4 cDNA used as an estradiol-independent mRNA control is the cDNA for human acidic ribosomal phosphoprotein PO. Nucl. Acids Res., 19 [symbol] 3998.

Lefebvre, A.-M., Peinado-Onsurbe, J., Leitersdorf, I., Briggs, M. R., Paterniti, J. R., Fruchart, J.-C., Fievet, C., Auwerx, J. and Staels, B. (1997) Regulation of lipoprotein metabolism by thiazolidinediones occurs through a distinct, but complementary mechanism relative to fibrates. Arterioscler.Thromb.Vasc.Biol., 17, 1756-1764.

Miller, M. J., Chotinaruemol, S., Sadowska-Krowikca, H., Zhang, X. J., McIntyre, J. A. and Clark, D. A. (1993) Guinea pig ileitis is attenuated by the leumedin N-(fluorenyl-9- methoxycarbonyl)-leucine (NPC 15199). J Pharmacol Exp Ther 266,468-472.

Nagy, L., Tontonez, P., Alvarez, J. G., Chen, H. and Evans, R. M. (1998) Oxidized LDL [symbol] regulates macrophage gene expression through ligand activation of PPARγ. Cell [symbol] 93, 229-240.

Oberfield, J. L., Collins, J. L., Holmes, C. P., Goreham, D. M., Cooper, J. P., Cobb, J. E. [symbol] Lenhard, J. M., Hull-Ryde, E. A., Mohr, C. P., Blanchard, S. G., Parks, D. J. [symbol] Moore, L. B., Lehmann, J. M., Plunket, K., Miller, A. B., Milbum, M. V., Kliewer [symbol] S. A. and Willson, T. M. (1999) A peroxisome proliferator-activated receptor [symbol] gamma ligand inhibits adipocyte differentiation. Proc Natl Acad Sci U S A, 96 [symbol] 6102-6106.

Rangwala, S. M., O'Brien, M. L., Tortorella, V., Longo, A., Loiodice, F., Noonan, D. J [symbol] and Feller, D. R. (1997) Stereoselective effects of chiral clofibric acid analogs on rat peroxisome proliferator-activated receptor alpha (rPPAR alpha) activation and peroxisomal fatty acid beta-oxidation. Chirality, 9, 37-47.

Rogniaux, H., Van Dorsselaer, A., Barth, P., Biellmann, J. F., Barbanton, J., van Zandt [symbol] M., Chevrier, B., Howard, E., Mitschler, A., Potier, N., Urzhumtseva, L., Moras [symbol] D. and Podjarny, A. (1999) Binding of aldose reductase inhibitors: correlation o [symbol] crystallographic and mass spectrometric studies. J Am Soc Mass Spectrom, 10 [symbol] 635-647.

Schoonjans, K. and Auwerx, J. (2000) Thiazolidinediones: an update. Lancet, 355 [symbol] 1008-1010.

Schoonjans, K., Martin, G., Staels, B. and Auwerx, J. (1997) Peroxisome proliferator [symbol] activated receptors, orphans with ligands and functions. Curr. Opin. Lipidol., [symbol] 159-166.

Schoonjans, K., Peinado-Onsurbe, J., Lefebvre, A. M., Heyman, R., Briggs, M., Deet [symbol] S., Staels, B. and Auwerx, J. (1996) PPARα and PPARγ activators direct [symbol] tissue-specific transcriptional response via a PPRE in the lipoprotein lipase gene [symbol] EMBO J., 15, 5336-5348.

Spiegelman, B. M. and Flier, J. S. (1996) Adipogenesis and obesity: rounding out th [symbol] big picture. Cell, 87, 377-389.

Stenson, W. F., Cort, D., Rodgers, J., Burakoff, R., DeSchryver-Kecskemeti, K [symbol] Gramlich, T. L. and Beeken, W. (1992) Dietary supplementation with fish oil in [symbol] ulcerative colitis. Ann. Intern. Med, 116, 609-614.

Su, G. C., Wen, X., Bailey, S. T., Jiang, W., Rangwala, S. M., Keilbaugh, S. A., [symbol] Flanigan, A., Murthy, S., Lazar, M. A. and Wu, G. D. (1999) A novel therapy for [symbol] colitis utilizing PPARgamma ligands to inhibit the epithelial inflammatory [symbol] response. J. Clin. Invest., 104, 383-389.

Vu-Dac, N., Schoonjans, K., Kosykh, V., Dallongeville, J., Fruchart, J.-C., Staels, B [symbol] and Auwerx, J. (1995) Fibrates increase human apolipoprotein A-II expression [symbol] through activation of the peroxisome proliferator-activated receptor. J. Clin. [symbol] Invest., 96, 741-750.

Willson, T. M., Brown, P. J., Sternbach, D. D. and Henke, B. R. (2000) The PPAR [symbol] s [symbol] from orphan receptors to drug discovery. J Med Chem, 43, 527-550.

Willson, T. M., Cobb, J. E., Cowan, D. J., Wiethe, R. W., Correa, I. D., Prakash, S. R [symbol] Beck, K. D., Moore, L. B., Kliewer, S. A. and Lehmann, J. M. (1996) The structure [symbol] activity relationship between peroxisome proliferator activated receptor [symbol] agonism and the antihyperglycemic activity of thiazolidinediones. J. Me [symbol] Chem., 39, 665-668.

Wright, H. M., Clish, C. B., Mikami, T., Hauser, S., Yanagi, K., Hiramatsu, R., Serha [symbol] C. N. and Spiegelman, B. M. (2000) A synthetic antagonist for the peroxisom [symbol] proliferator-activated receptor gamma inhibits adipocyte differentiation. J Bio [symbol] Chem, 275, 1873-1877.

Young, P. W., Buckle, D. R., Cantello, B. C., Chapman, H., Clapham, J. C., Coyle, P. J [symbol] Haigh, D., Hindley, R. M., Holder, J. C., Kallender, H., Latter, A. J., Lawri [symbol] K. W. M., Mossakowska, D., Murphy, G. J., Roxbee Cox, L. and Smith, S. [symbol] (1998) Identification of high-affinity binding sites for the insulin sensitiz [symbol] rosiglitazone (BRL49653) in rodent and human adipocytes using [symbol] radioiodinated ligand for peroxisomal proliferator-activated receptor gamma. [symbol] Pharmacol Exp Ther, 284, 751-759.

Yu, K., Bayona, W., Kallen, C. B., Harding, H. P., Ravera, C. P., McMahon, G., Brown, [symbol] M. and Lazar, M. A. (1995) Differential Activation of peroxisome Proliferator [symbol] activated receptors by eicosanoids. J. Biol. Chem., 270, 23975-23983.

1. A method for treating or preventing a PPAR-γ mediated disease or condition comprising administration of a therapeutically effective amount of a compound having the formula I: 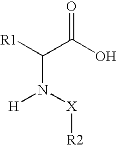
wherein R1 is selected from a linear or branched alkyl, alkenyl and alkynyl group comprising from 1 to 6 carbon atoms,
X is a chain comprising from 1 to 6 carbon atoms which may comprise one to four heteroatoms,
R2 is a condensed polycyclic group comprising at least two cycles.

2. A method according to claim 1 wherein the R2 group comprises at least two cycles selected from carbocycles and heterocycles.

3. A method according to one of claims 1 and 2 wherein the X chain comprises one or two carbon atoms which may be substituted by an oxo group.

4. A method according to one of claims 1 to 3 wherein R2 is a polycyclic group selected from 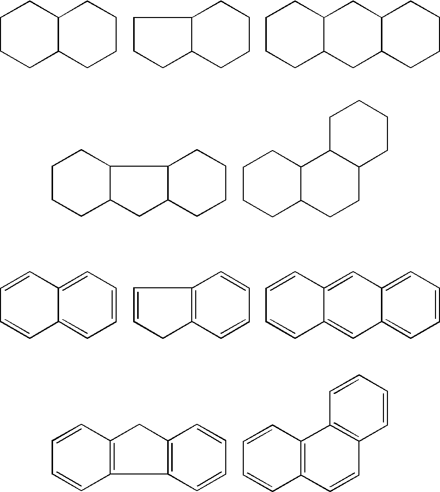
wherein said groups optionally comprise one to four heteroatoms selected from halogens, N, O and S.

5. A method according to claim 1 comprising administration of a therapeutically effective amount of a compound the formula I, wherein said compound is 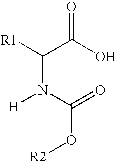
wherein R1 is selected from a linear or branched alkyl, alkenyl and alkynyl group comprising from 1 to 6 carbon atoms,
R2 is a polycyclic group selected from 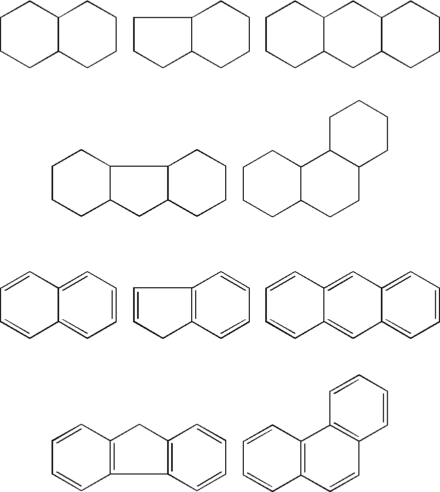
wherein said groups optionally comprise one to four heteroatoms selected from halogens, N, O and S.

6. A method according to claim 1 comprising administration of a therapeutically effective amount of a compound the formula I, wherein said compound is 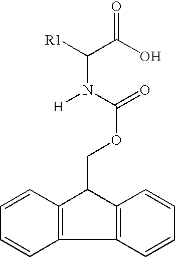
wherein R1 is selected from a linear or branched alkyl, alkenyl and alknyl group comprising from 1 to 6 carbon atoms and
wherein the said tricyclic group optionally comprises one to four heteroatoms selected from halogens, N, O and S

7. A method according to claim 1 comprising administration of a therapeutically effective amount of a compound the formula I, wherein said compound is 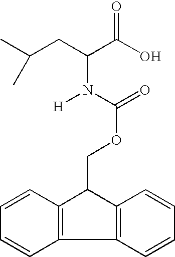
wherein the said tricyclic group optionally comprises one to four heteroatoms selected from halogens, N, O and S

8. A method according to claim 1 comprising administration of a therapeutically effective amount of N-(9-fluroroenyimethyloxycarbonyl)-L-Leucine.

9. A method according to one of claims 1 to 8 wherein said disease or condition is anorexia.

10. A method according to one of claims 1 to 8 for increasing or decreasing body weight.

11. A method according to one of claims 1 to 8 for increasing insulin sensitivity.

12. A method according to one of claims 1 to 8 for treating or preventing insulin resistance, as occurs in diabetes.

13. A method according to one of claims 1 to 8 wherein said disease or condition is a chronic inflammatory disorder.

14. A method according to one of claims 1 to 8 wherein said disease or condition is inflammatory bowel disease, ulcerative colitis or Crohn's disease.

15. A method according to one of claims 1 to 8 wherein the said disease or condition is arthritis, notably rheumatoid arthritis, polyarthritis and asthma.

16. A method according to one of claims 1 to 8 wherein said disease is cancer.

17. A method according to one of claims 1 to 8 wherein said disease is atherosclerosis.

18. A method according to one of claims 1 to 8 wherein said disease is a skin disorder, notably psoriasis.

19. A method according to one of claims 1 to 8 wherein said disease is hyperlypidemia.
